(19) United States
(12) Patent Application Publication
(10) Pub. No.: US 2006/0193783 A1
(43) Pub. Date: Aug. 31, 2006

(54) Low dose corticosteroid composition

(76) Inventors: Balaram Subhas Bhowmick, Akota (IN); Nitin Ritu Laddha, Akota (IN); Jaysingh Ajay Khopade, Akota (IN)

Correspondence Address: MERCHANT & GOULD PC, P.O. BOX 2903, MINNEAPOLIS, MN, 55402-0903, US

(21) Appl. No.: 10/545,305

(22) PCT Filed: Feb. 16, 2004
(86) PCT No.: PCT/IN04/00042

§ 371 Date: Aug. 11, 2005

(30) Foreign Application Priority Data
Feb. 17, 2003 (IN) 197/MUM/2003
Dec. 12, 2003 (IN) 1264/MUM/2003

(51) Int. Cl. A61K 31/724; A61K 31/573; A61L 9/04

(52) U.S. Cl. 424/45; 514/58; 514/171

(57) ABSTRACT

The present invention relates to a low dose composition of budesonide suitable for administration of budesonide to mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis comprising budesonide at a therapeutically effective dose of less than 16 mcg and a pharmaceutically acceptable liquid carrier.

The present invention relates to a low dose corticosteroid composition of budesonide said composition being suitable for administration of budesonide to mucosal membranes.

Particularly the present invention relates to a low dose composition of budesonide suitable for administration of budesonide to mucosal membranes for the management of nasal symptoms associated with seasonal allergic, rhinitis, perennial allergic rhinitis, perennial non-allergic rhinitis, nasal polyps, as well as prevention of post surgical polyps, chronic sinusitis and recurrent sinusitis comprising budesonide at a therapeutically effective dose of less than 16 mcg and a pharmaceutically acceptable liquid carrier.

The preferred low dose composition of budesonide suitable for administration of budesonide to the mucosal membranes is a stable aqueous solution composition of budesonide.

### BACKGROUND OF THE INVENTION

Budesonide, a synthetic corticosteroid, is an antiinflammatory agent useful for prophylaxis and treatment of asthma, and for treatment of perennial and seasonal allergic rhinitis. It is available in the form of an oral inhalation powder (PIMICORT® TURBUHALER, 200 mcg), and oral inhalation suspension (PULMICORT® RESPULES, 0.25 mg & 0.5 mg) for maintenance treatment of asthma and as prophylactic therapy. For rhinitis, it is available as a metered-dose nasal spray formulation (RHINICORT AQUA, 32 mcg) containing budesonide in aqueous suspension and a metered-dose pressurized aerosol unit (RINOCORT Nasal Inhaler) containing a suspension of micronized budesonide in a mixture of propellants, (dichlorodifluromethane, trichloromonofluromethane and dichlorotetrafluoroethane) and sorbitan trioleate.

Rhinocort nasal inhaler is a metered-dose pressurized aerosol unit, wherein each actuation releases 50 mcg budesonide from the valve and delivers approximately 32 mcg budesonide from the nasal adapter. The recommended starting dose is 256 mcg daily, given as either two sprays in each nostril morning and evening and as four sprays in each nostril in the morning. Rhinocort aqua, nasal spray 32 mcg, is a metered-dose, manual pump spray formulation, whose recommended starting dose for adults and children 6 years of age and older is 64 mcg per day administered as one spray per nostril once daily. The maximum recommended dose for adults is 256 mcg per day and for pediatrics is 128 mcg per day.

The Rhinocort aqua, nasal spray 32 mcg, is a metered-dose, manual-pump spray formulation containing a micronized suspension of budesonide in an aqueous medium. Systemic corticosteroid effects such as hypercorticism and adrenal suppression, has been reported to occur with the administration of larger doses of corticosteroids and hence it is recommended to use minimal effective dose of budesonide nasal spray. As mentioned above, for Rhinocort aqua, the recommended starting dose is 64 mcg per day administered as one spray per nostril of the available 32 mcg Nasal spray once daily which may be increased if the patients do not achieve the symptom control but in order to reduce the possibility of side effects it is always desirable to titrate an individual patient to the minimum effective dose.

We have now surprisingly found that a lower metered spray dose of budesonide than that previously recommended is effective for the management of nasal symptoms associated with allergic rhinitis and is also well tolerated when administered intranasaly. Thus we provide a composition comprising budesonide at a therapeutically effective dose of less than 16 mcg and pharmaceutically acceptable liquid carrier such that the composition is suitable for administration of budesonide to the mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis.

United States Application No. US20020016315A1 discloses a metered unit dose comprising 40 mcg or less of budesonide, its formulations and its use for the treatment of conditions in the nose. The patent exemplifies a suspension comprising 32 mcg budesonide. Studies on the marketed preparation Rhinocort® Nasal spray shows that a 32 mcg suspension is efficacious. There is no study or publication demonstrating that doses less than 16 mcg may be also efficacious. We have found novel compositions comprising budesonide at a therapeutically effective dose of less than 16 mcg that are effective in management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis thus meeting the much needed requirement of providing a less dose of a corticosteroid for therapeutic efficacy to reduce the possibility of side effects.

Thus a low dose composition of budesonide wherein the composition is suitable for administration of budesonide to mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis wherein the composition comprises of budesonide at a therapeutically effective dose of less than 16 mcg and pharmaceutically acceptable liquid carrier is provided in the present invention.

Further we have also found an aqueous pharmaceutically acceptable liquid carrier for budesonide wherein budesonide is present in dissolved form and when stored at room temperature for one year there is no precipitation of budesonide over the one-year period. More particularly we also found stable aqueous solution composition of budesonide which are free of alocoholic cosolvents suitable for administration of budesonide to mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis such that the composition is efficacious and well tolerated when administered at a metered spray dose of less than 16 mcg per nostril.

Further we have also found an aqueous pharmaceutically acceptable liquid carrier for budesonide wherein budesonide is present in dissolved form and when stored at room temperature for one year there is no precipitation of budesonide over the one-year period. More particularly we also found stable aqueous solution composition of budesonide which are free of alocoholic cosolvents suitable for administration of budesonide to mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis such that the composition is efficacious and well tolerated when administered at a metered spray dose of less than 16 mcg per nostril.

Budesonide, because of its high lipophilicity, is virtually insoluble in water. Hence, there are considerable difficulties in formulating aqueous solutions of budesonide including the need for the addition of solvents like alcohols, to keep the drug in solution. It is however desirable to avoid the use of alcoholic cosolvents. For example in a study reported in Ann Allergy 1988, Oct;(4):305-310, wherein the study compared the incidence of nasal burning and stinging, as well as overall tolerability of the currently marketed formulation of Rhinalar (flunisolide, marketed formulation) to a new formulation of Rhinlar containing less propylene glycol, the results showed a very significant difference in terms of severity, duration and tolerability in favour of the new formulation. Moreover the effects of long-term use of preparations containing substantial amount of a cosolvent are not known. Also, U.S. Pat. No. 6,241,969 has stated that there are limits to acceptable levels of cosolvents included in inhaled products and the limits are set by the propensity of these solvents either to cause local irritation of lung tissue and/or to intoxicate the patient. However, the problem associated with aqueous solutions of budesonide free of alcoholic cosolvent is that the drug undergoes decomposition, and because of its low solubility in water, it has a tendency to precipitate on storage at room temperature. It is thus a further object of the present invention to provide stable aqueous solution composition of budesonide characterized in that the composition is free from an alcoholic solvent and when stored at room temperature for one year exhibits no precipitation of budesonide. The said aqueous composition comprises budesonide and a complexing agent and has a pH not exceeding 6.0. Various techniques used in prior art to develop stable budesonide solutions are given below.

Patentees in U.S. Pat. No. 5,914,122 disclosed that although prior to their invention budesonide aqueous solutions could be prepared with the help of organic water-soluble alcohols, in these solutions budesonide decomposed within a short time and thus prior to the invention ready to use stable solution preparations of budesonide were not known. The patent claims stable budesonide solution with pH less than 6.0 wherein budesonide is dissolved in a solvent selected from the group consisting of water, alcohol, and a water/alcohol mixture and wherein the alcohol is selected from ethanol, isopropanol and propylene glycol. Alcohol free aqueous solution compositions of budesonide that were stable with respect to chemical decomposition of budesonide as well as free from precipitation of budesonide upon storage at room temperature were not disclosed. The patent further claims that the composition further comprises stabilizing additives selected from sodium EDTA (0.001%-0.1% w/w) and cyclodextrin (0.05%-1.0% by weight).

U.S. Pat. No. 6,241,969 claims aerosolized composition of a corticosteroid to respiratory tract consisting essentially of 5 mcg/ml to 5 mg/ml of the corticosteroid, about 0.1 to about 20% by weight of a high-HLB surfactant component comprising at least 50% by weight of an ethoxylated derivative of vitamin E and at least about 70% by weight of an aqueous phase. All the supportive examples of the composition, however, illustrate compositions that comprise both a high-HLB surfactant as well as a cosolvent like propylene glycol, ethanol, polyethylene glycol etc. Budesonide is one of the corticosteroids further claimed in this patent. The high-HLB surfactant component exemplified and claimed is tocopheryl polyethylene glycol 1000 succinate. The invention further discloses addition of excipients such as buffers, osmotic agents, low toxicity antifoaming agents and preservatives. When buffers are used for pH adjustment, the recommended range is of pH within 4-8 and more preferably within 5-6.8. The patented composition does not contain any complexing agent that complexes budesonide.

United States Application No. US20020031558A1 claims clear aqueous solutions of bile acids, bile acid derivatives, bile salts or conjugated bile acids, which remain in solution over range of pH values. These aqueous compositions were useful when orally administered for treating gastric and peptic ulcer disease, liver disease, gall stones and hyperlipidemia The invention thus relates to the therapeutic potential of bile acid, salts, derivatives or conjugates. However it claims in dependent claims also clear solutions as defined above further comprising pharmaceutically effective compounds. The only example supporting this further claim uses bismuth citrate as the pharmaceutically effective compound. According to the claims, the clear aqueous solution may also be administered intranasally. Although budesonide is one of the pharmaceutically effective compounds the patent does not exemplity such compositions and therefore does not address issues such as chemical stabilization of budesonide, or prevention of precipitation of budesonide over a period of time of storage. The disclosed compositions do not contain a complexing agent capable of complexing with budesonide.

PCT Application WO 01/07014 claims a stable formulation for inhalation through nebulization consisting of a solution of a steroid in which (a) the steroid concentrations ranges from 0.01%-0.1%; (b) the carrier is a mixture of water and propylene glycol in a ratio ranging from 60:40 to 30:70 v/v; and (c) pH is adjusted with concentrated strong acid in a range from 3.5 to 5.0; wherein the percentage of nebulized active ingredient particles with mean aerodynamic diameter below 6 μm is higher than 70% and the nebulization efficiency is higher than 20%. Budesonide or its epimers are further claimed as one of the steroids. The inventors found that the solutions adjusted to pH 4 and 4.5 remained stable. The inventors further studied stability of the budesonide solutions by adjusting the pH using buffers consisting of different relative percentages of sodium phosphate and citric acid buffers. They concluded that inclusion of buffers resulted in less stable solutions. A high concentration of an alcoholic solvent like propylene glycol used in the disclosed formulation is undesirable for use on mucosal membranes. Russian Patent No. RU2180217 discloses a stable budesonide solution with an anti-inflammatory effect that contains budesonide, propylene glycol, polyethylene oxide, benzoic acid, Trilon B, nipazole, thiourea and water. The solution can additionally contain 2-hydroxypropyl-beta-cyclodextrin.

PCT Publication No. WO 01/87203 claims a stable budesonide solution comprising budesonide and a solvent wherein after about 12 weeks, budesonide is present in solution in an amount at least about 90% preferably at least about 95%, most preferably at least about 99% of the initial budesonide concentration in the solution. The solvent used was generally a non-aqueous solvent such as polyhydric alcohol or glycol, more preferably propylene glycol or triethylene glycol but water could be added as a cosolvent.

U.S. Pat. No. 6,491,897 claims a stable pharmaceutical budesonide aerosol solution with a pH between 2.0 and 7.0 in which the budesonide is dissolved in a water/ethanol mixture, which mixture optionally includes an alcohol selected from the group consisting of isopropanol and propylene glycol and wherein the stable solution comprises 0.001% to 5% by weight of budesonide. All the disadvantages quoted above with regard to use of alcoholic cosolvents in nasal and pulmonary delivery systems will apply to this patented composition.

U.S. Pat. No. 4,383,992 claims a water-soluble inclusion compound formed by complexing beta-cyclodextrin with a steroid compound having a molecular structure smaller than the inside dimension of the internal cavity of beta-cyclodextrin. Another independent claim claims, an anti-inflammatory pharmaceutical preparation comprising the above inclusion compound mixed with non-toxic pharmaceutical carrier. The example in this patent, teaches the method of forming the inclusion compound of beta-cyclodextrin and the steroids (like, prednisolone acetate or dexamethasone or hydrocortisone) wherein dispersing agent like hydroxypropyl methylcellulose has also been added and a solution obtained. The patent however does not disclose inclusion complex formation of budesonide with beta-cyclodextrin and whether it would provide a stable formulation. Nor does it address issues such as the pH requirement for stabilization of budesonide solutions or the prevention of precipitation of budesonide over a period of time of storage.

### OBJECTS OF THE INVENTION

The objects of the invention are
1) To provide a low dose corticosteroid composition of budesonide suitable for administration of budesonide to mucosal membranes.
2) To provide a low dose composition of budesonide suitable for intranasal administration of budesonide for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis wherein the composition comprises of budesonide at a therapeutically effective dose of less than 16 mcg per nostril.
3) To provide a composition as in 2 above wherein the composition comprises budesonide and pharmaceutically acceptable liquid carrier wherein the liquid carrier is aqueous.
4) To provide an aqueous solution composition of budesonide which is free of alcoholic solvents.
5) To provide an aqueous solution composition of budesonide which is substantially free of a cosolvent.
6) To provide aforesaid aqueous solution composition of budesonide wherein budesonide is stabilized against its chemical decomposition.
7) To provide aforesaid aqueous solution composition where instead of solubilising budesonide with alcoholic cosolvents, the solubilization is achieved by the use of a complexing agent capable of forming a complex with budesonide.
8) To also provide aforesaid aqueous solution composition as in 7 above wherein further the propensity of budesonide to precipitate on long-term storage is inhibited.
9) To provide aqueous solution composition of budesonide having a low local irritation of mucosal membranes.
10) To provide aqueous solution composition of budesonide having a high availability of budesonide at the site of action.
11) To provide an aqueous solution composition of budesonide having a high availability at the site of action and reduced systemic availability of budesonide or its metabolites.
b 12) To provide an aqueous solution composition of budesonide as in 11 above and having a low dose of budesonide.
13) To provide a method of treatment for management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis by administering budesonide intranasaly at a therapeutically effective dose of less than 16 mcg per nostril.
14) To provide a method of treatment for management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis by administration of a stable aqueous solution comprising budesonide in a dissolved form.

### SUMMARY OF THE INVENTION

Thus the present invention provides a low dose composition of budesonide suitable for administration of budesonide to mucosal membranes.

The present invention particularly provides a low dose composition of budesonide suitable for administration of budesonide to mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis wherein the composition comprises of budesonide at a therapeutically effective dose of less than 16 mcg and a pharmaceutically acceptable liquid carrier.

The present invention more particularly provides a low dose composition of budesonide wherein the composition is a stable aqueous solution composition of budesonide said composition being suitable for administration of budesonide to mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis such that the composition is efficacious and well tolerated when administered at metered spray dose of less than 16 mcg per nostril.

The said composition comprises budesonide and pharmaceutically acceptable liquid carrier wherein the liquid carrier is aqueous and budesonide is present in dissolved form and when stored at room temperature for one year there is no precipitation of budesonide over the one-year period. Particularly the said composition is a stable aqueous solution composition comprising budesonide in an aqueous medium, which is free from an alcoholic cosolvent wherein the medium comprises a complexing agent wherein, said aqueous solution has a pH not exceeding 6.0

Thus a method of treatment for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis in a mammal in need thereof which comprises intranasal administration of budesonide at a therapeutically effective dose of less than 16 mcg per nostril is provided herewith in the present invention.

### DETAILED DESCRIPTION OF THE PRESENT INVENTION

According to the invention we provide a low dose composition comprising budesonide and pharmaceutically acceptable liquid carrier.

We particularly provide an aqueous solution composition of budesonide which is effective when administered at a metered spray dose of less than 16 mcg per nostril said composition being suitable for nasal administration to a mammal in a single dose for the treatment of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis (both persistent and intermittent).

The therapeutically effective dose may be from 2 mcg to less than 16 mcg per nostril, preferably from 5 mcg to less than 16 mcg per nostril and more preferably from about 8 mcg to about 15 mcg per nostril.

By administering the solution composition of budesonide at such a low metered spray dose, it is possible for the patients to take a lower metered daily dose, which is not only effective for treatment of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis but is also well tolerated and safe to the nose. The solution composition administered at this dose is also convenient to the patients and thus provides added advantage of better patient compliance.

Accordingly we provide a therapeutic method of treating nasal symptoms associated with seasonal allergic rhinitis, perennial allergic rhinitis, perennial non-allergic rhinitis, nasal polyps, as well as prevention of post surgical polyps, chronic sinusitis and recurrent sinusitis by administering into each nostril of a mammal a metered spray dose of less than 16 mcg of a solution composition of budesonide. The dose is administered to each nostril from once-a-day to up to eight times in a day; preferably administered as a metered spray dose of less than 16 mcg per nostril per day. More preferably the solution composition is sprayed in each nostril at a dose of 15 mcg/spray once-a-day to achieve the desired effective dose of 30 mcg/day.

The stable aqueous solution composition of budesonide is a clear stable aqueous solution administered as intranasal drops or intranasal sprays wherein the recommended lower dose can be administered from a drop pipette or a rhinyl or from nasal inhaler or a spray device. The dose can be administered from unit dose containers or from metered dose spray pumps with varying dose volumes, wherein the concentration of budesonide in the composition is adjusted to give the desired unit dose of budesonide.

In the stable aqueous solution composition of the present invention the budesonide is solubilized in water by complexation with a complexing agent. Preferable the complexing agent is chemical species capable of forming an inclusion complex with budesonide. Preferred inclusion complex forming agents are cyclodextrins. The cyclodextrins that may be used in the present invention is any of the water-soluble substituted or unsubstituted cyclodextrins or its derivatives thereof which can be well tolerated when administered by nasal, pulmonary or oral route, which is for e.g. α, β- or γ-cyclodextrins or derivatives thereof, preferably derivatives wherein one or more of the hydroxy groups are substituted, e.g. by alky, hydroxyalkyl, carboxyalkyl, alkylcarbonyl, carboxyalkoxyakyl, alkylcarbonyloxyalkyl, alkoxycarbonylalkyl or hydroxy-(mono or polyalkoxy)alkyl groups, wherein each alkyl or alkylene moiety preferably contains up to six carbons. Substituted cyclodextrins, which can be used in the present invention, include ethers, polyethers or mixed ethers thereof. More preferably such substituted cyclodextrins are ethers wherein the hydrogen of one or more cyclodextrin hydroxy groups is replaced by one or more cyclodextrin hydroxy groups is replaced by C₁₋₃alkyl, hydroxy-C₂₋₄malkyl or carboxy-C₁₋₂allyl or more particularly by methyl, ethyl, hydroxyethyl, hydroxypropyl, hydroxybutyl, carboxymethyl or carboxyethyl. Most preferred cyclodextrins used in the present invention are β-cyclodextrin ethers and polyethers e.g. dimethyl-β-cyclodextrin, hydroxypropyl- β-cyclodextrin and hydroxyethyl- β-cyclodextrin. In the aqueous solution composition of the present invention, the cyclodextrin is preferably used at 0.05% w/v to about 15.0% w/v, more preferably at 1.0% w/v to about 10.0% w/v and most preferably at 1.5% w/v to about 5.5% w/v.

In preferred embodiments of the stable aqueous solution composition of present invention hydroxypropyl-β-cyclodextrin is used. Commercially, Hydroxypropyl-β-cyclodextrin is made by reacting β-cyclodextrin with propylene oxide, it has a true density of 1.378 g/cm³ and has a cavity diameter of 6.0-6.5 Å.

The pH of the stable aqueous solution composition of the present invention may be adjusted to less than 6, preferably in the range between about 6.0 and 3.5, more preferably between, 4.0 and 4.5. Examples of suitable pH adjusting agents, selected from a group of buffering agents, comprises lactic acid, citric acid, tartaric acid, phosphoric acid, acetic acid, hydrochloric acid, nitric acid, sodium or potassium metaphosphate, sodium or potassium phosphate, sodium or potassium acetate, ammonia, sodium carbonate, sodium or potassium hydroxide, dibasic sodium phosphate, sodium borate, and the like and mixtures thereof. Adjusting pH using strong mineral acids like hydrochloric acid is more preferred.

A precipitation inhibitor may be included in the stable aqueous solution composition of the present invention. Precipitation inhibitor used in the stable aqueous solution composition of present invention that prevents the precipitation of budesonide from the solution comprises of a primary precipitation inhibitor comprising a water soluble hydrophilic polymer optionally in mixture with an auxillary precipitation inhibitor comprising solubilizing agents selected from a) cosolvents other than an alcoholic solvent and b) solubilizers selected from anionic, cationic, and/or non-ionic surfactant or mixtures thereof.

The water-soluble hydrophilic polymers used as primary precipitation inhibitor in the stable aqueous solution composition of the present invention includes and are not restricted to cellulose and cellulose derivatives, vinyl pyrrolidone polymers or polyvinylpyrrolidone and polyvinyl alcohol. Examples of cellulose and cellulose derivatives that can be used include hydroxypropyl cellulose (HPC), hydroxypropyl methylcellulose (HpMC), carboxymethyl cellulose (CMC) and the like. Various grades of hydroxypropyl methylcellulose may be used in the present invention.

The grades available are categorized depending upon the chemical substitution and hydration rates. Hydroxypropyl methylcellulose having % methoxy content of 19-24% and hydroxypropyl content of 7-12% with a fastest relative rate of hydration is available commercially under the brand name of Methocel Grade K. Hydroxypropyl methylcellulose with 28-30% methoxy content and 7-12% of hydroxypropyl content with a faster relative hydration rate as compared to the above grade is available commercially under the brand name of Methocel Grade E. Hydroxypropyl methylcellulose with 27-30% methoxy content and 4-7.5% of hydroxypropyl content with a slow relative hydration rate is available as Methocel F grade and that with 27.5-31.5% methoxy content and 0% hydroxypropyl content and with slowest rate of hydration is available as Methocel Grade A. In preferred embodiments of the aqueous solution composition of the present invention, Hydroxypropyl methylcellulose with a viscosity ranging from about 3500 mPas to about 5600 mPas of a 2% w/v aqueous solution, commercially available as Methocel E4 is used as the stabilizing primary precipitation inhibitor in an amount ranging from about 0.01% w/v to about 5.0% w/v. Even very small quantities of the water-soluble hydrophilic polymer serve to achieve a beneficial precipitation inhibiting effect in the stable aqueous solution composition of the present invention and thus more preferably it is used in an amount ranging from about 0.1% w/v to about 0.5% w/v.

Any mucosally non-irritant cosolvent may be used as an auxiliary precipitation inhibitor. The cosolvent is used in low quantities and the composition is free from substantial quantity of the cosolvent. A composition that is substantially free of cosolvent and uses less than 5% cosolvent, preferably less than 2% cosolvent is preferred. The more preferred cosolvent is N-methyl pyrrolidone. In preferred embodiments, budesonide may be first dissolved in this dipolar aprotic water-miscible broad-spectrum solubilizer, N-methyl-pyrrolidone, used in an amount just enough to dissolve the drug, i.e in an amount ranging from 0.1% w/v to about 2.0% w/v and this drug solution may be added to the cyclodextrin solution and mixed intimately to form the inclusion complex. N-methyl-pyrrolidone has a viscosity of 1.65 mPas at 25° C. and is known to increase solubility, rate of solubilzation and solution stability of drugs in aqueous solutions. Additionally, when N-methyl pyrrolidone is used nasally it does not cause any nasal irritation or does not change the nasal volume.

Further the stable aqueous solution composition of the present invention may contain other conventional pharmaceutical excipients, e.g. osmogens, chelating agents, preservative agents, antioxidants etc.

Examples of the osmogent(s) that may be used in the stable aqueous solution composition of the present invention include all pharmaceutically acceptable and pharmacologically inert water-soluble compounds referred to in the pharmacopoeias such as United States Pharmacopoeia, as well as in Remington: The Science and Practice of Pharmacy; edition 19; Mack Publishing Company, Easton, Pa. (1995). Pharmaceutically acceptable water-soluble salts of inorganic or organic acids, or non-ionic organic compounds with high water solubility, e.g. carbohydrates such as sugar, or amino acids, are generally preferred. The examples of agents used for rendering the solution isoosmotic include inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; sodium carbonate or sodium bicarbonate; carbohydrates such as minitol, sorbitol, arabinose, ribose, xylose, glucose, dextrose, fructose, mannose, galactose, sucrose, maltose, lactose, raffinose, inositol, xylitol, maltitol; water-soluble amino acids such as glycine, leucine, alanine, or methionine; urea and the like, and mixtures thereof. Preferred osmogent is dextrose, which may be added in an amount, which renders the solution isoosmotic.

Chelating agents that may be used in the stable aqueous solution composition of the present invention are selected from a group comprising edetic acid, edetic acid salts like disodium edetate, sodium edetate, edetate calcium disodium and trisodium edetate, malic acid and the like and mixtures thereof.

A preservative agent may be added in the stable aqueous solution composition of the present invention to protect the solution composition from microbial contamination. Examples of such preservatives are benzalkonium chloride, methyl paraben, propyl paraben and their salts, potassium sorbate and sodium benzoate. The preservatives may be present in an amount ranging from about 0.002 to 0.5% w/w of the formulation. The preferred preservative used in the present invention is potassium sorbate.

The stable aqueous solution composition of the present invention may be prepared by a process wherein budesonide and a complexing agent are mixed in an aqueous medium free from an alcoholic cosolvent to form a solution, other conventional pharmaceutically acceptable excipients are added and the pH is adjusted to less than 6.0. The obtained solutions according to the process may be filled in suitable containers such as multidose vials or preferably in monodose vials and used in forms such as and not restricted to nasal drops, nasal sprays, metered aerosol, inhalation sprays and the like. The solution compositions may be sterilized when required by any techniques used in art, preferably by filtration. Additionally, it is preferred that during the manufacturing process exposure to oxygen or oxidative materials, or light be avoided to protect the solution from any potentially ensuing damage.

In a preferred embodiment, the stable aqueous solution composition of the present invention may be prepared by a simple process comprising dissolving budesonide in N-methyl pyrrolidinone and mixing with aqueous dispersion of hydroxypropyl methylcellulose and hydroxypropyl β- cyclodextrin. Dextrose, disodium edetate and potassium sorbate is dissolved in water for injection and mixed with the budesonide solution mixture. Under stirring, the pH of the solution is adjusted using 5% v/v hydrochloric acid to pH 4.0-4.5 and volume made up with water for injection. The solution may be filtered under nitrogen pressure to obtain a clear stable aqueous solution composition of budesonide.

The solution composition of budesonide was administered to patients to evaluate its efficacy and tolerability in comparison to a suspension composition of budesonide, the results of which is discussed in example 5 given below.

The invention is illustrated by the following examples, which is by no means intended to limit the scope of the invention but is given by way of illustration.

### EXAMPLE 1

This example illustrates one embodiment of the present invention and a process for its preparation, formula for which is given in Table 1 below.

TABLE 1

| Ingredients | % w/v | mg/10 ml |
|---|---|---|
| Budesonide | 0.064 | 6.4 |
| Hydroxypropyl | 0.250 | 25 |
| methylcellulose | | |
| (Methocel E4M) | | |
| Hydroxypropyl | 5.5 | 550 |
| β-cyclodextrin | | |
| Dextrose | 1.6 | 16 |
| (anhydrous) | | |
| N-methyl | 1.0 | 100 |
| pyrolidone | | |
| Disodium edetate | 0.1 | 10 |
| Potassium sorbate | 0.12 | 12 |
| Hydrochloric acid | qs to pH 4.0-4.5 | qs to pH 4.0-4.5 |
| (1% v/v) | | |
| Water for | qs to 100% | qs to 10 ml |
| injection | | |

Hydroxypropyl methylcellulose was dispersed in about 40% of water for injection heated to 80° C., chilled 25% water for injection was then added to it after cooling the above solution to 40° C. Hydroxypropyl β-cyclodextrin dissolved in water for injection was added to the the above formed slightly viscous clear solution under stiring. Budesonide dissolved in N-methyl pyrrolidinone was added dropwise under stirring to the above solution. The dextrose, disodium edetate and potassium sorbate was dissolved in 20% water of injection and added to the drug solution. pH of the resultant solution was adjusted using hydrochloric acid between 4.0-4.5 and volume made up with the remaining water for injection. The solution was filtered through 0.22 micron nylon 66 membrane filter using a pre-filter of 2 micron glass fibre and filled into vials.

The solutions were subjected to stability studies by storing in sealed vials at 40° C./75% RH and 25° C./60% RH storage conditions. The initial and 3 months data for the samples are given in Table 2 below.

TABLE 2

| | | description | | Assay | Total |
|---|---|---|---|---|---|
| conditions | Months | (*) | pH | (% content) | RS** |
| Initial | — | complies | 4.01 | 100.36 | 0.48 |
| 40° C./75% RH | 1 | complies | 3.82 | 96.38 | 1.42 |
| | 3 | complies | 3.82 | 90.14 | 4.65 |
| 25° C./60% RH | 1 | complies | 3.83 | 98.24 | 0.75 |
| | 3 | complies | 3.80 | 94.55 | 1.60 |

*complies means complies with the specification and the specification is-clear solutions without any presipitation.
**total RS is total related substances.

### EXAMPLE 2

This example illustrates another embodiment of the present invention.

TABLE 3

| Ingredients | % w/v | mg/10 ml |
|---|---|---|
| Budesonide | 0.064 | 6.4 |
| Hydroxypropyl | 0.2 | 20 |
| methylcellulose | | |
| (Methocel E4) | | |
| Hydroxypropyl | 2.5 | 250 |
| β-cyclodextrin | | |
| N-methyl | 1.0 | 100 |
| pyrollidinone | | |
| Disodium edetate | 0.1 | 10 |
| Potassium sorbate | 0.12 | 12 |
| Hydrochloric acid | qs to pH 4.5-5.0 | qs to pH 4.0-4.5 |
| (1% v/v) | | |
| Water for | qs to 100% | qs to 10 ml |
| injection | | |

Hydroxypropyl methylcellulose was dispersed in about 40% of water for injection heated to 80° C., chilled 25% water for injection was then added to it after cooling the above solution to 40° C. Hydroxypropyl β-cyclodextrin dissolved in water for injection was added to the the above formed slightly viscous clear solution under stirring. Budesonide dissolved in N-methyl pyrrolidinone was added dropwise under stirring to the above solution. The disodium edetate and potassium sorbate was dissolved in 20% water of injection and added to the drug solution. pH of the resultant solution was adjusted using hydrochloric acid between 4.0-4.5 and volume made up with the remaining water for injection. The solution was filtered through 0.22 micron nylon 66 membrane filter using a pre-filter of 2 micron glass fibre and filled into vials.

### EXAMPLE 3

This example Mustrates another embodiment of the present invention.

TABLE 4

| Ingredients | % w/v | mg/10 ml |
|---|---|---|
| Budesonide | 0.128 | 12.8 |
| Hydroxypropyl | 0.25 | 25 |
| methylcellulose | | |
| (Methocel E4) | | |
| Hydroxypropyl | 5.0 | 500 |
| β-cyclodextrin | | |
| N-methyl | 1.2 | 120 |
| pyrollidinone | | |
| Disodium edetate | 0.1 | 10 |
| Potassium sorbate | 0.12 | 12 |
| Hydrochloric acid | qs to pH 4.0-4.5 | qs to pH 4.0-4.5 |
| (1% v/v) | | |
| Water for | qs to 100% | qs to 10 ml |
| injection | | |

The process for the preparation of the solution composition was same as that given in example 2.

### EXAMPLE 4

This example illustrates another embodiment of the present invention.

TABLE 5

| Ingredients | % w/v | mg/10 ml |
|---|---|---|
| Budesonide | 0.128 | 12.8 |
| Hydroxypropyl | 0.25 | 25 |
| methylcellulose | | |
| (Methocel E4) | | |
| Hydroxypropyl | 5.5 | 550 |
| β-cyclodextrin | | |
| Dextrose | 1.5 | 150 |
| N-methyl | 1.2 | 120 |
| pyrollidinone | | |
| Disodium edetate | 0.1 | 10 |
| Potassium sorbate | 0.12 | 12 |
| Hydrochloric acid | qs to pH 4.0-4.5 | qs to pH 4.0-4.5 |
| (1% v/v) | | |
| Water for | qs to 100% | qs to 10 ml |
| injection | | |

The process for the preparation of the solution composition was same as that given in example 1.

The solutions were subjected to stability studies by storing in sealed vials at 40° C./75% RH and 25° C./60% RH storage conditions. The initial and 3 months data for the samples are given in Table 6 below.

TABLE 6

| | | description | | Assay | Total |
|---|---|---|---|---|---|
| conditions | Months | (*) | pH | (% content) | RS** |
| Initial | — | complies | 4.00 | 99.36 | 0.45 |
| 40° C./75% RH | 1 | complies | 3.70 | 96.52 | 1.37 |
| | 3 | complies | 3.67 | 94.58 | 2.36 |
| 20° C./60% RH | 1 | complies | 3.82 | 97.61 | 0.96 |
| | 3 | complies | 3.70 | 97.55 | 1.37 |

*complies means complies with the specification and the specification is-clear solution without any presipitation.
**total RS is total related substances.

### EXAMPLE 5

This example illustrates another embodiment of the present invention.

TABLE 7

| Ingredients | % w/v | mg/10 ml |
|---|---|---|
| Budesonide | 0.030 | 3.0 |
| Hydroxypropyl | 0.25 | 25 |
| methylcellulose | | |
| (Methocel E4) | | |
| Hydroxypropyl | 5.5 | 550 |
| β-cyclodextrin | | |
| Dextrose | 1.6 | 160 |
| N-methyl | 1.0 | 100 |
| pyrollidinone | | |
| Disodium edetate | 0.1 | 10 |
| Potassium sorbate | 0.12 | 12 |
| Hydrochloric acid | qs to pH 4.0-4.5 | qs to pH 4.0-4.5 |
| (1% v/v) | | |
| Water for | qs to 100% | qs to 10 ml |
| injection | | |

The process for the preparation of the solution composition was same as that given in example 1.

### EXAMPLE 6

A randomized, parallel-group, multi-center, double-blind, comparative study was performed to evaluate the efficacy and tolerability of two formulations of budesonide nasal spray in allergic rhinitis (persistent or intermittent).

Eighty adults and adolescents (individuals at least 12 years of age) were randomly allocated to either the test formulation of budesonide nasal spray 30 mcg/day (15 mcg/spray in each nostril once-a-day) or reference formulation of budesonide nasal spray 64 mcg/day (32 mcg/spray in each nostril once-a-day).

The formulations were evaluated for primary efficacy (mean change from baseline in total nasal symptom scores (TNSS) and total non-nasal symptom scores (TNNSS) were assessed on day 8, 15, 22 and 29, relief of individual symptoms (viz. sneezing, rhinorrhoea, nasal itching, congestion, ocular itch/burning, watering/tearing, redness of eye), tolerability and overall global assessment in order to determine their clinical efficacy and safety.

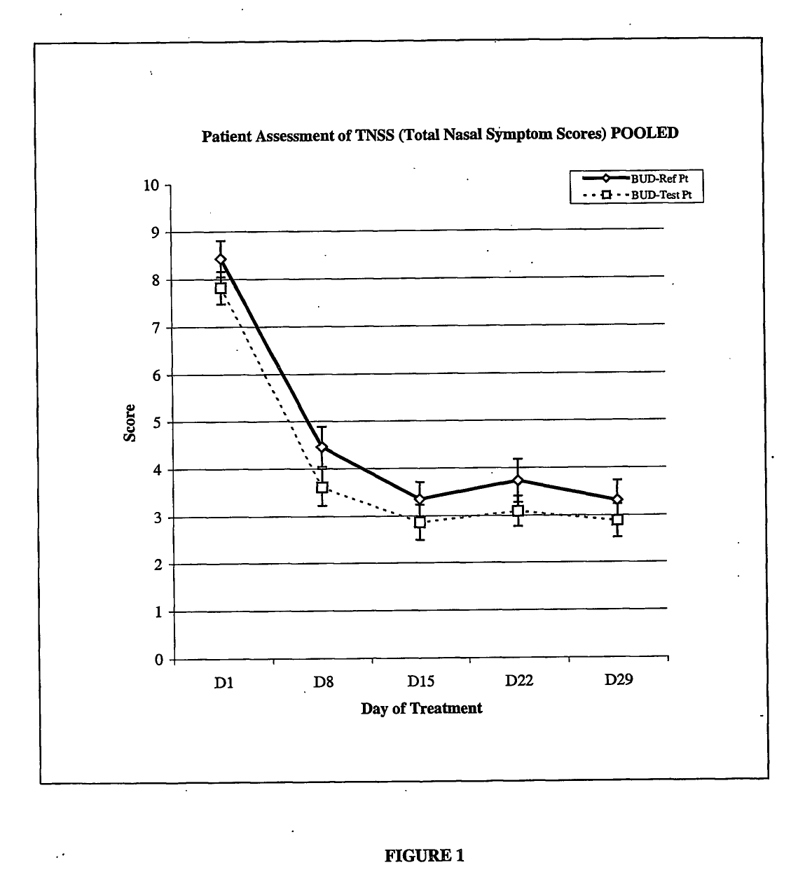

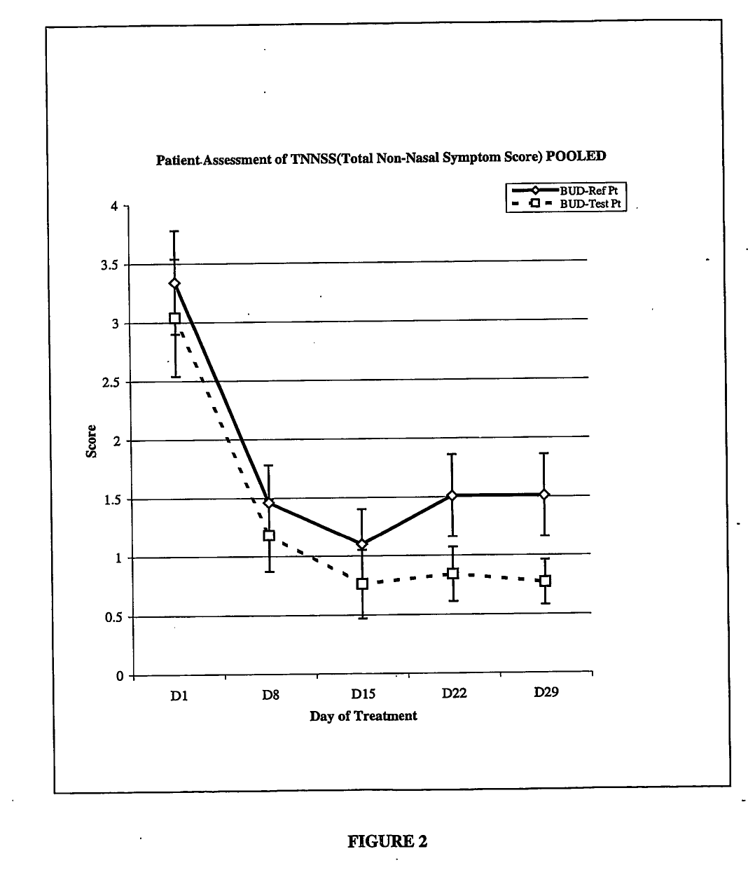

The patient assessment of total nasal symptom score is graphically demonstrated in FIG. 1 and the non-nasal symptom score is demonstrated in FIG. 2.

The results of the study demonstrated that budesonide when administered as an aqueous solution composition was effective in controlling the symptoms of allergic rhinitis at a much lower sprayed dose of 15 mcg/spray in each nostril once-a-day in comparison to a suspension composition of budesonide which demonstrated similar effect at a much higher dose of 32 mcg/spray in each nostril once-a-day. Both the formulations were well tolerated with no side effects.

1. A low dose corticosteroid composition comprising budesonide and pharmaceutically acceptable liquid carrier suitable for administration of budesonide to the mucosal membranes for the management of nasal symptoms of seasonal allergic rhinitis, perennial allergic rhinitis, perennial non-allergic rhinitis, nasal polyps, as well as prevention of post surgical polyps, chronic sinusitis and recurrent sinusitis comprising budesonide at a therapeutically effective dose of less than 16 mcg

2. A composition as claimed in claim 1 wherein the therapeutically effective dose is from 5 mcg to less than 16 mcg.

3. A composition as claimed in claim 2 wherein the therapeutically effective dose is from about 8 mcg to about 15 mcg.

4. A composition as claimed in claim 1 wherein the pharmaceutically acceptable liquid carrier is aqueous, the budesonide is present in dissolved form and when stored at room temperature for one year there is no precipitation of budesonide over the one year period.

5. A composition as claimed in claim 4 wherein the stable aqueous solution composition comprises budesonide and a complexing agent capable of forming a complex with budesonide characterized in that the composition is free from an alcoholic cosolvent, has a pH not exceeding 6.0.

6. A composition as claimed in claim 5 wherein the complexing agent is a β-cyclodextrin ether or polyether selected from dimethyl-β-cyclodextrin, hydroxypropyl-β-cyclodextrin, hydroxyethyl- β-cyclodextrin and the like and mixtures thereof.

7. A composition as claimed in claim 6 wherein the β-cyclodextrin used is hydroxypropyl- β-cyclodextrin.

8. A composition as claimed in claim 7 wherein the hydroxypropyl-β-cyclodextrin is used in an amount ranging from about 0.05% w/v to about 15.0% w/v.

9. A composition as claimed in claim 8 wherein the hydroxypropyl-β-cyclodextrin is used in an amount ranging from about 1.0% w/v to about 10.0% w/v.

10. A composition as claimed in claim 9 wherein the hydroxypropyl-β-cyclodextrin is used in an amount ranging from about 1.5% w/v to about 5.5% w/v.

11. A composition as claimed in claim 5 further comprising a precipitation inhibitor that prevents the precipitation of budesonide from the solution.

12. A composition as claimed in claim 11 wherein the precipitation inhibitor comprises of a primary precipitation inhibitor comprising a water soluble hydrophilic polymer, optionally in mixture with an auxillary precipitation inhibitor comprising solubilizing agents selected from a) cosolvents other than an alcoholic solvent and b) solubilizers selected from anionic, cationic, and/or non-ionic surfactant or mixtures thereof.

13. A composition as claimed in claim 12 wherein the primary precipitation inhibitor comprising a water-soluble hydrophilic polymer is selected from a group of cellulose and cellulose derivatives, vinyl/poly vinyl pyrrolidone polymers, polyvinyl alcohol or mixtures thereof.

14. A composition as claimed in claim 13 wherein the cellulose and cellulose derivatives is selected from hydroxypropyl cellulose (HPC), hydroxypropyl methylcellulose (HPMC), carboxymethyl cellulose (CMC) and the like.

15. A composition as claimed in claim 12 wherein the primary precipitation inhibitor is hydroxypropyl methylcellulose.

16. A composition as claimed in claim 15 wherein the hydroxypropyl methylcellulose is having a viscosity in the range from 3500 mPas to about 5600 mPas of a 2% w/w aqueous solution.

17. A composition as claimed in claim 12 wherein the primary precipitation inhibitor is used in an amount ranging from about 0.01% w/v to about 5.0% w/v.

18. A composition as claimed in claim 12 wherein the cosolvent other than alcoholic solvent is N-methyl pyrrolidone.

19. A composition as claimed in claim 5 which further comprises a chelating agent.

20. A composition as claimed in claim 19 wherein the chelating agent is selected from a group comprising edetic acid, edetic acid salts like disodium edetate, sodium edetate, edetate calcium disodium and trisodium edetate, malic acid and the like and mixtures thereof.

21. A composition as claimed in claim 5 wherein further dextrose is used as an osmogent.

22. A composition as claimed in claim 5 wherein further potassium sorbate is used as a preservative.

23. A composition as claimed in claim 5 wherein the aqueous solution having a pH not exceeding 6.0 comprises a buffering agent.

24. A composition as claimed in claim 23 wherein the buffering agent is selected from a group comprising lactic acid, citric acid, tartaric acid, phosphoric acid, acetic acid, hydrochloric acid, nitric acid, sodium or potassium metaphosphate, sodium or potassium phosphate, sodium or potassium acetate, ammonia, sodium carbonate, sodium or potassium hydroxide, dibasic sodium phosphate, sodium borate, and the like and mixtures thereof.

25. A method of treatment for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis in a mammal in need thereof which comprises intranasal administration of budesonide at a therapeutically effective dose of less than 16 mcg per nostril.

26. A method of treatment as claimed in claim 25 wherein the therapeutically effective dose is from 5 mcg to 16 mcg.

27. A method of treatment as claimed in claim 26 wherein the therapeutically effective dose is from about 8 mcg to about 15 mcg.

28. A method of treatment as claimed in claim 25 wherein the dose is administered to each nostril from once-a-day to up to eight times in a day.

29. A method of treatment as claimed in claim 25 wherein the dose is administered to each nostril once-a-day.

30. A method of treatment as claimed in claim 25 wherein budesonide is administered in a composition such that it is suitable for administration of budesonide to the mucosal membranes for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis of seasonal or perennial allergic rhinitis.

31. A method of treatment as claimed in claim 30 wherein the composition is a stable aqueous solution composition.

32. A method of treatment for the management of nasal symptoms associated with seasonal allergic rhinitis, perennial allergic rhinitis, perennial non-allergic rhinitis, nasal polyps, as well as prevention of post surgical polyps, chronic sinusitis and recurrent sinusitis by administering a stable aqueous solution comprising budesonide in a dissolved form.

33. (canceled)

34. (canceled)

35. (canceled)

36. (canceled)

37. (canceled)
